(19) United States
(12) Patent Application Publication
(10) Pub. No.: US 2011/0319717 A1
(43) Pub. Date: Dec. 29, 2011

(54) OPTICAL WINDOW FOR IN VIVO IMAGING DEVICE

(76) Inventor: Amit PASCAL, Haifa (IL)

(21) Appl. No.: 12/822,572

(22) Filed: Jun. 24, 2010

(51) Int. Cl. A61B 1/06; B23P 11/00

(52) U.S. Cl. 600/177; 29/428

(57) ABSTRACT

An optical system for preventing shaded areas and method of manufacturing the optical system include an imager, at least one illumination source, and an optical window, behind which the imager and the at least one illumination source are positioned. The optical window may include a diffusing element on a portion thereof, for scattering illumination passing through it, such that light scattered from that portion of the window illuminates an otherwise under-illuminated region of impingement of the illumination. The portion on which the diffusing element is located, is positioned outside a field of view of the imager and within a field of illumination of the at least one illumination source.

### FIELD OF THE INVENTION

The present invention relates to the field of in-vivo imaging devices' optical systems, more specifically to optical systems that are designed to prevent shaded areas.

### BACKGROUND OF THE INVENTION

Known devices may provide in-vivo sensing, such as imaging or sensing of other in-vivo parameters. Autonomous in-vivo sensing devices, such as, for example, swallowable or ingestible capsules or other devices may move through a body lumen, sensing as they move along. An autonomous in-vivo sensing device such as an imaging device may include, for example, an imager for obtaining images from inside a body cavity or lumen, such as the gastrointestinal (GI) tract. The imager may, for example, be associated with an optical system, and optionally a transmitter and an antenna. Illumination units, such as LEDs, may be used to illuminate the in-vivo lumen. In some of these devices, the imager and illumination source are both located behind a single window and the lumen is illuminated and imaged through the same window.

### SUMMARY OF THE INVENTION

Embodiments of the present invention may provide an optical system which may comprise an imager; at least one illumination source; and an optical window, for example, having a dome shape. In some embodiments, the imager and at least one illumination source are positioned behind the optical window. In some embodiments of the invention, the optical window comprises a diffusing element on a portion of the window, in order to scatter illumination passing through the portion for diverting at least a portion of the light towards a shaded area.

According to some embodiments of the present invention, the diffusing element may be for scattering illumination passing through a portion of the window in a substantially homogenous manner. The diffusing element may be on a portion of the window located outside a field of view of the imager but within a field of illumination of at least one illumination source. The diffusing element may include a high roughness area on at least one surface of at least the portion of the window. For example, the window may have a roughness average of between 1 nm and 1 μm on both surfaces of the window, this being a typical smoothness that is regarded as providing a transparent window in the visible and NIR, and the high roughness area may have a roughness average of above 5 μm on at least one surface of the portion of the window.

According to some other embodiments of the present invention, an optical system may comprise: an imager; at least one illumination source; and an optical window, for example, having a dome shape. In some embodiments, the imager and at least one illumination source are positioned behind the optical window. In some embodiments of the invention, the optical window comprises an optical element to direct illumination through the window in a predetermined direction. According to some embodiments, the optical element that diverts the illumination in a predetermined direction may include a lens, a prism a DOE (Diffractive Optical Element) or any other suitable optical element. The optical element may be located outside a field of view of the imager but within the field of illumination of the at least one illumination source.

Further, embodiments of the present invention may provide a method of manufacturing an optical window for an optical system to prevent shaded areas. The method may comprise determining an area in the window where little or substantially no light is passing through; and inserting an optical element into the window for directing illumination towards a shaded area.

According to some embodiments of the present invention, the optical element may shift the illumination so as to compensate for the deviation angle which the light would have been deviated at, in the absence of such an optical element. The optical element may direct light such that the angle in which the illumination exits the window is substantially the same as the angle of illumination, in order to prevent lack of illumination in a certain portion of the tissue, for example in a portion of the tissue positioned opposite to the portion of the window that includes the optical element.

According to some other embodiments of the present invention, the insertion of the optical element may include production of a diffusing element on a portion of the window, for scattering the light passing through this portion of the window so that at least a portion of the light may be directed towards a shaded area caused by the refraction. The diffusing element may be produced on a portion of the window located within a field of illumination of the illumination sources but outside a field of view of the imager, for example, in order to prevent distortions in the image acquired by the imager. The diffusing element may scatter the light passing through this portion of the window in a substantially homogenous manner. The production of the diffusing element may include the process of roughening at least one surface of at least a portion of the window.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic diagram of a system for in-vivo imaging according to some embodiments of the present invention;

FIG. 2A is a schematic illustration of an optical system for in-vivo imaging, illustrating a problem which may be solved by embodiments of the present invention;

FIG. 2B is a schematic illustration of an optical system for in-vivo imaging according to embodiment of the invention;

FIG. 3 is a flowchart illustrating a method for manufacturing an optical window according to embodiments of the present invention; and

FIG. 4 is an illustration of an exemplary illumination distribution on an imager's field of view in prior art imaging devices.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In the following detailed description, numerous specific details are set forth in order to provide a thorough understanding of the present invention. However, it will be understood by those skilled in the art that the present invention may be practiced without these specific details. In other instances, well-known methods, procedures, and components have not been described in detail so as not to obscure the present invention.

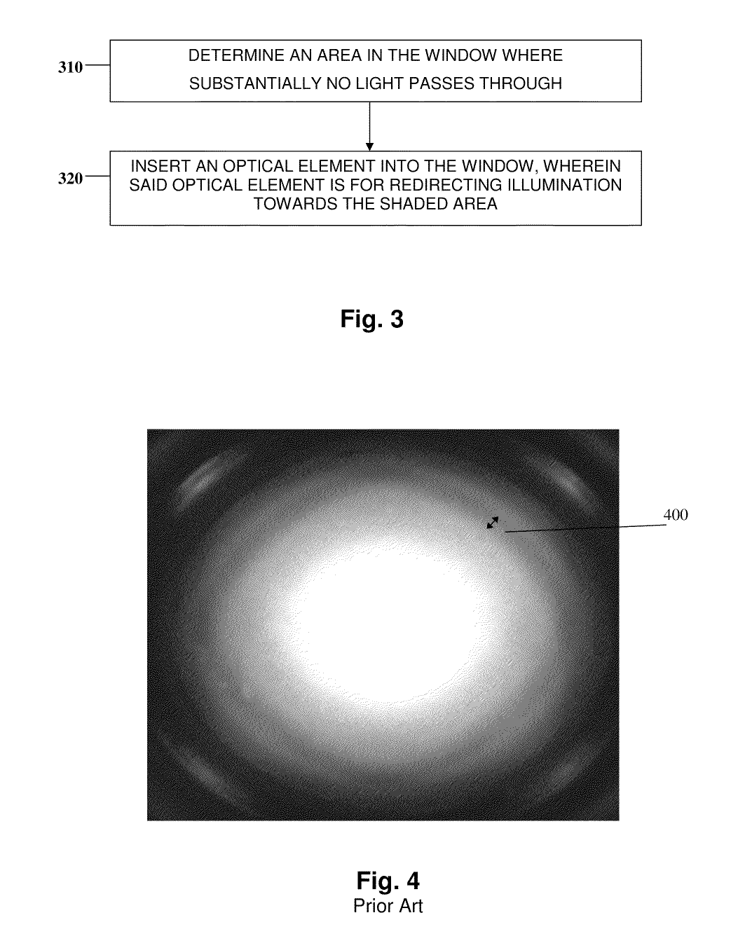

In some in-vivo imaging devices, the imager and illumination source are both located behind a single window and the lumen is illuminated and imaged through the same window. The window in such imaging devices may have a changing curvature, which in some cases may not change continuously. For example, a dome shaped window may have a convex shaped portion in front of the imager, and flat shaped portion at the sides of the imager. On portions of the window in which the shape of the window changes, for example, from convex to flat, the diversion of light may be greatly increased. When a broad angle of view is required, some regions of the body lumen may be located in less-illuminated regions due to the increased diversion of light. Therefore, a very small amount of light from the illumination sources may reach these regions of the body lumen. Therefore, shadows or dark areas may appear in the field of view of the imager. FIG. 4 illustrates an exemplary illumination distribution on an imager's field of view. As shown in FIG. 4, a dark ring 400 may be created at a certain circumferential region of the imager's field of view. Therefore, a portion of the body lumen tissue may not be sufficiently well captured by the imager.

Embodiments of the present invention provide an optical system and a method for manufacturing an optical system which may reduce or prevent shadows or dark areas which may appear in the field of view of the imager.

A system according to some embodiments of the invention may include an in-vivo sensing device transmitting information (e.g., images and/or other data) to a data receiver and/or recorder possibly close to or worn on a subject. A data receiver and/or recorder may of course take other suitable configurations. The data receiver and/or recorder may transfer the received information to a larger computing device, such as a workstation or personal computer, where the data may be further analyzed, stored, and/or displayed to a user. In other embodiments, each of the various components need not be required and/or may be housed in alternate configurations; for example, an internal device may transmit or otherwise transfer (e.g., by wire) information directly to a viewing or processing system. In another example, the data receiver or workstation may transmit or otherwise transfer information to the in-vivo device.

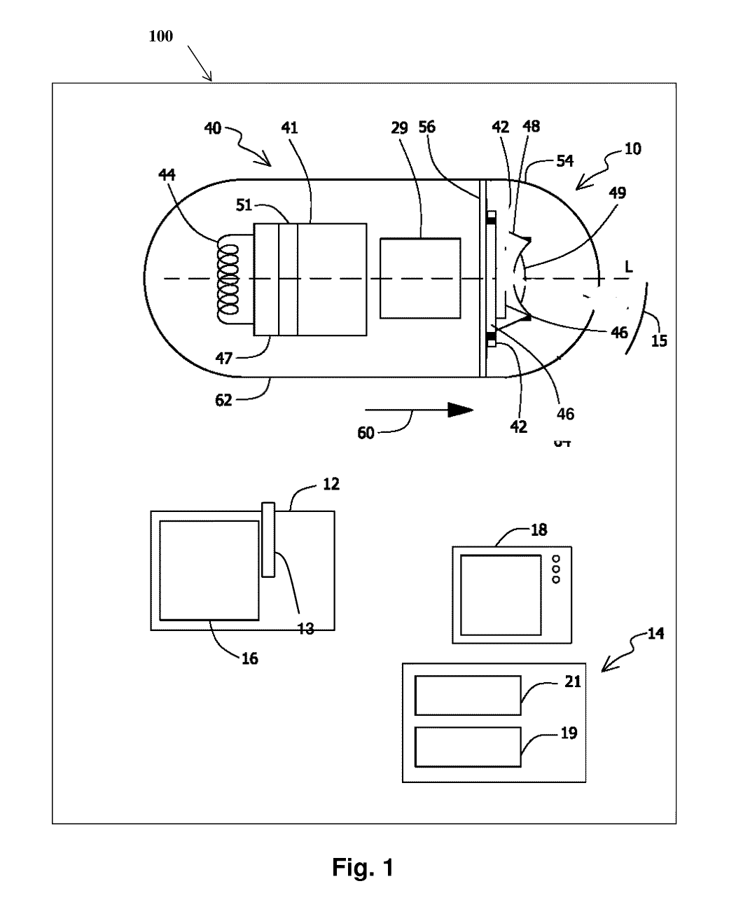

It is noted that some embodiments of the present invention may be directed to an autonomous, typically ingestible in-vivo device. Other embodiments need not be ingestible and/or autonomous. Devices or systems according to embodiments of the present invention may be similar to the imaging system illustrated in FIG. 1 and the corresponding text as described in U.S. Patent Application Publication No. 2007/0118018 which is assigned to the common assignee of the present invention and which is hereby fully incorporated herein by reference. Devices according to embodiments of the present invention may further be similar to PillCam™ SB or PillCam™ ESO2 imaging devices, which are available from Given Imaging LTD., Yoqneam, Israel. Furthermore, a receiving system suitable for use with embodiments of the present invention may be similar to Data Recorder DR2.0C, available from Given Imaging LTD., which may receive signals from a swallowable in-vivo imaging device (e.g. PillCam™ ESO2) as the device passes through the GI tract. Display systems and/or software suitable for use with embodiments of the present invention may be similar to the RAPID® software also available from Given Imaging LTD, which enables reviewing images captured by a swallowable imaging device, e.g. PillCam™ ESO2. Devices and systems as described herein may have other configurations and other sets of components.

Reference is made to FIG. 1, which is a schematic diagram of a system 100 for in-vivo imaging according to some embodiments of the present invention. In one embodiment, system 100 may include an in-vivo imaging device 40 which may, for example, be capsule shaped. Device 40 may include an optical system which includes a dome shaped optical viewing window 54, which may typically define a space 52 behind which are positioned components such as one or more lens 49, lens holder or separator 48, an imager 46, one or more illumination source(s) 42 such as, for example, Nichia model number 0012 LED available from NICHIA Corporation, or any other suitable LED model. The imaging device 40 also includes one or more power source(s) 29, which may include, for example, a suitable battery such as, for example, a silver oxide Energizer battery or Varta model V399 available from VARTA AG, for long recording period or model V370 for short period or any other suitable Battery model. Alternatively or additionally, power source 29 may include other devices, such as a unit for receiving power from an external source. Window 54 may be a protective optical window, preferably made of plastic such as thermoplastic polyurethane resins, polymethyl, methacrylate, cyclic olefin copolymer, polycarbonates or other suitable plastic, glass or ceramic transparent material. Typically, the imager 46 images through window 54 and illumination source(s) 42 illuminates through window 54. Lens holder 48 may include an aperture (not shown) through which light reflected from, for example, an object 15, may be received. Device 40 may include a transmitter 41 (typically operating wirelessly via radio waves) which may include a transceiver 51, and an antenna 44, for transmitting images and possibly other information to, for example, an external receiving device 12. Other types of transmitters and wireless or wired transmission methods may be used; for example, in an endoscope application, wire or other transmission may be used. In some embodiments, transmitter 41 may further include a processor 47, which may perform initial processing of images and possibly other information prior to the transmission of the images and/or other information.

According to some embodiments, imager 46 and illumination sources 42 may be fixed or otherwise attached to a substrate 56 which may include, for example, a circuit board. According to another embodiment of the invention, the various components of the device 40 may be disposed on a circuit board including rigid and flexible portions; preferably the components are arranged in a stacked vertical fashion. Substrate 56 may define a viewing direction 60 of device 40, wherein viewing direction 60 may be perpendicular to substrate 56.

In one embodiment, an imaging device may include more than one image sensor. For example, an additional optical system may be included in a direction opposite viewing direction 60, to form, for example, a double ended viewing device. Other configurations for including more than one imager 46 in device 40 and/or more than one viewing direction may be implemented.

Imager 46 may include, for example, a CCD camera or imager, a CMOS camera or imager such as, for example, the CMOS imager used in PillCam™ SB2 or PillCam™ ESO2, as provided by Given Imaging Ltd. of Yoqneam, Israel, or other suitable one or more imagers, cameras, receiving units or image acquisition components.

Typically, located outside the patient's body in one or more locations may be a data recorder 12, a data processor 14, and an image monitor 18. Data recorder 12 may typically include an antenna or antenna array 13 and a storage unit 16. Data processor 14 may include a processor 19 and a storage unit 21. Image monitor 18 may display, inter alia, images recorded by, for example, device 40. Typically, data processor 14 and monitor 18 may be part of a personal computer or workstation, which may include standard components such as a processor 19, a memory, a disk drive, and input-output devices, although alternate configurations are possible. Data processor 14 may typically, as part of its functionality, act as a controller controlling the display of the images. Image monitor 18 may typically be a conventional video display, but may, in addition, be any other device capable of providing images or other data and may be of any size monitor including large projection size monitors. The image monitor 18 may present the image data, typically in the form of still and/or a stream of image frames, and in addition may present other information. In an exemplary embodiment, the various categories of information may be displayed in windows. Other displaying formats may be used. In other embodiments of the present invention, one or more of the components included in receiver 12 and data processor and/or workstation 14 may be packaged in alternate configuration and may be or may be included in a portable or stationary device, package, and/or housing.

In operation, imager 46 may capture images and may send data representing the images to transmitter 41, which may transmit data to data recorder 12 using, for example, electromagnetic radio waves. Data recorder 12 may transfer the image data to storage unit 16. After a certain period of time of data collection, the image data stored in storage unit 16 may be transferred to the data processor 14 or the data processor storage unit 21. For example, data recorder 12 or storage unit 16 may be taken off the patient's body and may be connected to a personal computer or workstation that may include the data processor 14 via a standard data link, e.g., a serial, parallel, USB, or wireless interface. According to one embodiment the image data may then be transferred from the storage unit 16 to data processor storage unit 21. Data processor 14, including possibly dedicated software, may analyze the data and provide the analyzed data to the image monitor 18, where a user views the image data. Other configurations allow for real time viewing. Further, other methods of recording, transmitting, storing and viewing images recorded by imager 46 may be used.

As discussed in detail above, there may be areas on window 54 where illumination from illumination sources 42 may be refracted in a non homogeneous manner, such that there may be shaded areas on object 15. Typically such areas may be areas where the shape of window 54 changes. For example, when window 54 is dome shaped, at the areas where the dome changes from convex to flat, light rays may be refracted such that there may be substantially no or little light passing through those areas. This may cause shadow or lack of light on a portion of object 15 which is located in front of the areas in window 54 where the dome changes from convex to flat. According to some embodiments of the invention, portions of window 54 may include a diffusing element, discussed in detail below, which may deflect light towards the shadowed portion of object 15.

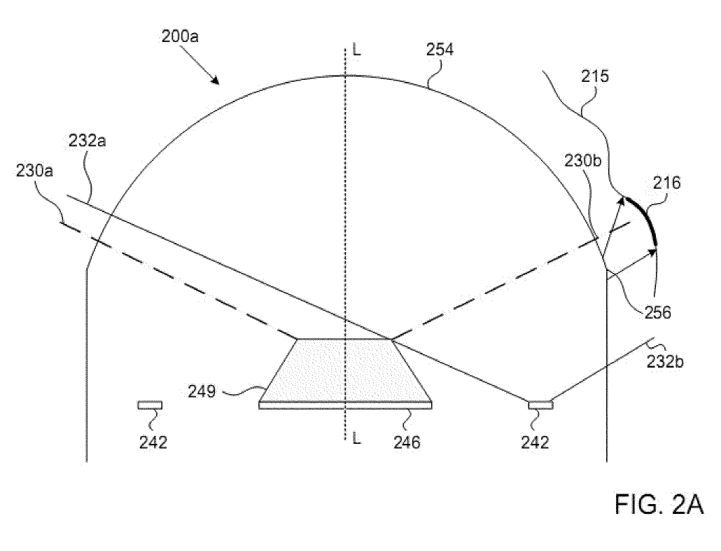

Reference is now made to FIG. 2A, which is a schematic illustration of an optical system 200a for in-vivo imaging, illustrating a problem which may be solved by embodiments of the present invention. Optical system 200a may include optical window or dome 254, illumination sources 242, lens holder 249 and imager 246.

Illumination sources 242 may illuminate an object or tissue 215 through dome 254. In some embodiments, the Field Of View (FOV) of imager 246 may be defined as the angle between lines 230a and 230b. The Field Of Illumination (FOI) as illustrated in FIG. 2A is the FOI of illumination source 242 which is positioned to the right of longitudinal axis L. The FOI of the illumination source 242 which may be positioned to the left of axis L is not marked in the drawing, however it would be symmetrical to the illustrated FOI. The illustrated FOI may be defined as the angle between illumination rays 232a and 232b, which are emitted from illumination source 242 located to the right of axis L. The illustrated FOI is restricted from one side (the side closer to axis L) by the height of lens holder 249. Lend holder 249 may block light from reaching the side of window 254 that is opposite the side where illumination source 242 (which is located to the right of axis L) is positioned, thereby dictating the location of ray 232a. From the other side of this illumination source 242 (i.e. the side closer to the window rather than to axis L), the FOI is restricted by the length of the transparent window 254 or by the location in which the optical window 254 is attached to a shell of an imaging device, e.g. device 40 described above with reference to FIG. 1. Since an imaging device's shell is typically opaque, the shell may block light from passing through the window 254 and illuminating tissue 215, thereby dictating the location of ray 232b. Illumination source(s) 242 may illuminate section 216 of object 215, among other object sections, through section 255 of window 254. While window section 255 may be outside the FOV of imager 246, it may be within the FOI of illumination source(s) 242.

In some embodiments, there may be areas of window 254 on which the light may be deviated in a substantial manner from its original direction. Those areas may typically be located, for example, in areas where the shape of window 254 changes. For example, when window 254 is dome shaped, at the areas where the dome's shape changes from convex to flat, light may be deviated considerably from its original direction on the internal surface of the window such that there may be substantially no or little light passing through those areas. This may cause shadow or lack of light on portions of tissue 215 which would have received the illumination if the angle in which the illumination exits window 254 was substantially the same as the original angle of illumination, such as, for example, portion 216 of tissue 215. Therefore, tissue section 216 may appear on imager 246 as a dark area such as for example, dark ring 400 shown in FIG. 4. The width of the shaded tissue section 216 depends on the distance between window 254 and tissue 215. As closer window 254 is to the tissue the smaller the shaded tissue section 216 is. When window 254 is farther away from tissue 215, the portion of shaded section 216 which is within the FOV is greater, so that the dark area may be more noticeable.

Reference is now made to FIG. 2B, which is a schematic illustration of an optical system 200b for in-vivo imaging according to embodiments of the present invention. Optical system 200b may be similar to optical system 10 described above with reference to FIG. 1. Optical system 200b may be included in an in-vivo imaging device similar to device 40 described above with reference to FIG. 1. Optical system 200b may include optical window or dome 254, illumination sources 242, lens holder 249 and imager 246.

In some embodiments, a section 256 of window 254 may include diffusing elements for transmitting light impinging on it uniformly towards tissue section 216, which may suffer from lack of illumination as discussed in detail above.

In some embodiments, the diffusing element may include increased roughness of section 256 of window 254. According to some embodiments, a surface of section 256 may be designed to have high roughness. For example, if window 254 may have an average surface roughness of between 1 nm and 1 μm, for example, on both internal and external surfaces, section 256 may be designed to have higher roughness average, for example, above 5 μm on at least one of its surfaces. The high roughness on section 256 may be achieved by sanding the mold of window 254, wherein the degree of roughness may be determined by the size of the sand particles and the duration of the treatment. Alternatively, the window itself may be etched or sand blasted in the appropriate regions. The specific degree of required roughness average on section 256 may depend, for example, on the optical arrangement behind the window.

Typically, the internal surface of section 256 is designed to have high roughness. However, in other embodiments the external surface of section 256 may be roughened, and in yet other embodiments, both the internal and external surfaces of section 256 may be roughened. Usually, the internal surface of window 254 is easier to roughen than the external surface. Additionally, a roughened internal surface may provide a better distribution of the illumination, because of the relatively large difference between the refractive indexes of the air inside window 254 and the plastic of which window 254 is usually made. Roughening of the external surface is also possible, although in this case the required roughness level may be higher in order to sufficiently scatter the illumination, because of the relatively small difference between the refractive indexes of the watery in vivo environment and the plastic of window 254. When light strikes a surface with high roughness, microscopic irregularities of the surface may scatter the light in irregular directions 236, which may correlate to a substantially uniform spread of illumination. Instead of light being refracted at a certain angle of refraction, light may be scattered to different directions 236, such that some of the illumination rays may reach section 216.

In some embodiments, the required degree of roughness and/or the location and/or the size of roughened section 256 of window 254 may depend on the shape of the window 254 and/or on the arrangement of illumination sources 242 within the window 254. Roughened section 256 should be located on a portion of window 254 which is included in the FOI of at least one of the illumination sources (in order to scatter the light provided by illuminations sources 242) but is outside of the FOV of the imager. This way, for example, the negative effect of the distortion of light by the roughened surface on the image quality will be reduced and/or avoided. Backscatter light which may be caused by the roughening may not enter the FOV and thus for example, may not affect the quality of image. Accordingly, different shapes of window 254 and different configurations of illumination sources 242 behind window 254, may require a different level/range of roughness on window 254 in order to deflect at least a portion of the illumination towards tissue section 216 with minimum negative effect on the image quality.

In other embodiments, additionally or alternatively to section 256 having a high roughness surface, system 200b may include an optical element for redirecting illumination. According to some embodiments of the present invention, the optical element may shift the illumination so as to compensate for the deviation angle which the light would have been deviated at in the absence of such an optical element, in order to prevent lack of illumination in a certain portion of the tissue, for example in a portion of the tissue positioned opposite to the portion of window 254 that includes the optical element, such as, for example, portion 216. Such optical elements for diverting illumination by a predetermined angle may be a lens, a prism, a DOE (Diffractive Optical Element) a pattern molded on window 254, a beam shaper covering the illumination source or other elements of the sort. An optical element included in window 254 may direct the light in a certain predetermined direction or in irregular directions as described above to ensure illumination on certain areas of object 215, for example, area 216. For example, additionally or alternatively to the roughened surface of section 256, system 200b may include an optical element which may divert the light such that the angle in which the illumination exits window 254 is substantially the same as the angle of illumination, thus, for example, illumination passing through section 256 may be focused onto section 216. Section 256 which includes the optical element that diverts the light should be located on a portion of window 254 which is included in the FOI of at least one of the illumination sources (in order to scatter the light provided by illuminations sources 242) but is outside of the FOV of the imager.

According to some embodiments, object 215 may be an in vivo area which an in vivo imaging device, such as device 40 as shown in FIG. 1, illuminates and acquires images of. Object 215 may be an in vivo tissue. Object 215 may typically be part of the gastrointestinal (GI) tract, which an in vivo imaging device such as device 40, may image as it passes through it.

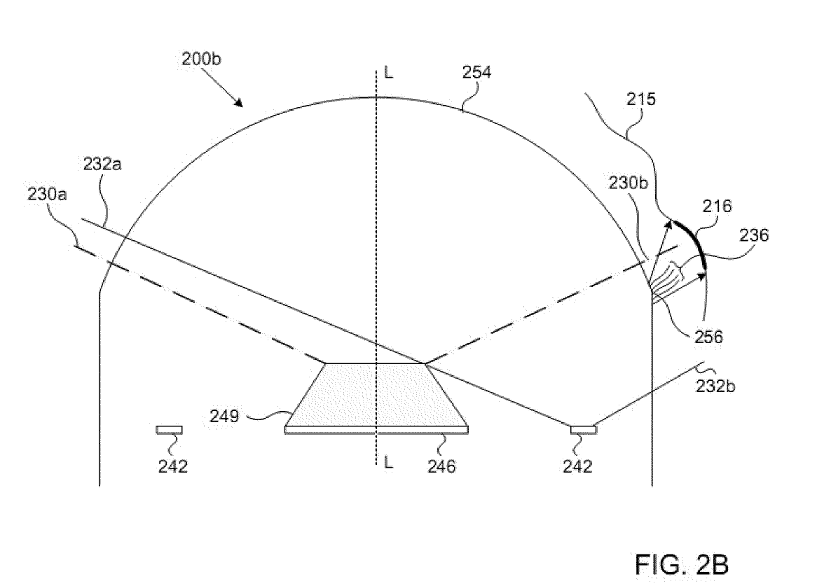

Reference is now made to FIG. 3, which is a flowchart illustrating an exemplary method for manufacturing an optical window according to embodiments of the present invention. As indicated in block 310, the method according to embodiments of the present invention may include determining an area in the window where substantially no light passes through, for example, because the light is considerably deviated on an internal surface of the window, thereby creating a shaded area in the window. As indicated in block 320, the method according to embodiments of the present invention may include inserting an optical element into the window, wherein said optical element is for redirecting illumination towards the shaded area.

According to some embodiments of the present invention, the optical element may redirect the illumination such that the angle of refraction is substantially similar to the angle of incidence.

According to some other embodiments of the present invention, the insertion of the optical element may include production of a diffusing element on a portion of the window, for scattering the light passing through this portion of the window in different directions, so that at least a portion of the light may be directed towards a shaded area caused by a change in the shape of the window. The diffusing element may be produced on a portion of the window located outside a field of view of the imager but within a field of illumination of the illumination sources. The diffusing element may scatter illumination passing through it, such that light scattered from the portion of the window which includes the diffusing element, illuminates an otherwise under-illuminated region of impingement of the illumination. The diffusing element may scatter the light passing through this portion of the window in a substantially homogenous manner. The producing of the diffusing element may include roughening at least one surface of at least a portion of said window.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. An optical system comprising:
an imager;
at least one illumination source; and
an optical window, behind which said imager and said at least one illumination source are positioned,
wherein said optical window comprises a diffusing element on a portion thereof, said portion located within a field of illumination of said at least one illumination source and outside a field of view of said imager, said diffusing element scattering illumination passing through it, such that light scattered from said portion of said window illuminates an otherwise under-illuminated region of impingement of said illumination.

2. The optical system according to claim 1, wherein said diffusing element is to scatter illumination passing through said portion of said window in a substantially homogenous manner.

3. The optical system according to claim 1, wherein said diffusing element is a high roughness area on at least one surface of at least said portion of said window.

4. The optical system according to claim 1, wherein said optical window is dome shaped.

5. The optical system according to claim 3, wherein said window has a roughness average of between 1 nm and 1 μm on both surfaces of said window, and said high roughness area has a higher roughness average of above 5 μm on at least one surface of said portion of said window.

6. The optical system according to claim 3, wherein said at least one surface is the internal surface.

7. The optical system according to claim 3, wherein said at least one surface is the external surface.

8. An optical system comprising:
an imager;
at least one illumination source; and
an optical window,
wherein said imager and at least one illumination source are positioned behind said optical window, said optical window comprising an optical element to direct illumination through the window in a predetermined direction, said optical element located within the field of illumination of said at least one illumination source and outside a field of view of said imager.

9. The optical system according to claim 8, wherein optical element is selected from a group consisting of: a lens, a prism and a DOE (Diffractive Optical Element).

10. The optical system according to claim 8, wherein said optical window is dome shaped.

11. A method of manufacturing an optical window for an optical system to prevent shaded areas, the method comprising:
determining an area in the window where substantially no light passes through, thereby creating a shaded area; and
inserting an optical element into the window, said optical element located within a field of illumination of at least one illumination source of said optical system and outside a field of view of an imager of said optical system, wherein said optical element is for redirecting illumination towards said shaded area.

12. The method according to claim 11, wherein said optical element is for redirecting illumination such that an angle of refraction is substantially similar to an angle of incidence.

13. The method according to claim 11, wherein said inserting comprises producing a diffusing element on a portion of said window, for scattering the light passing through said portion.

14. The method according to claim 13, wherein said producing comprises roughening at least one surface of at least said portion of said window.

15. The method according to claim 13, wherein said diffusing element is for scattering the light passing through said portion of said window in a substantially homogenous manner.
